(19) United States
(12) Patent Application Publication
(10) Pub. No.: US 2021/0059562 A1
(43) Pub. Date: Mar. 4, 2021

(54) DYNAMIC VITAL-SIGN DETECTION SYSTEM AND METHOD

(71) Applicant: Wistron Corporation, New Taipei City (TW)

(72) Inventor: Fang-Ming Wu, New Taipei City (TW)

(21) Appl. No.: 16/680,244

(22) Filed: Nov. 11, 2019

(30) Foreign Application Priority Data
Aug. 30, 2019 (TW) 108131423

(51) Int. Cl. A61B 5/08; A61B 5/05; A61B 5/00

(52) U.S. Cl. CPC A61B 5/0826; A61B 5/05; A61B 5/0816; A61B 5/7203; A61B 5/7225

(57) ABSTRACT

A dynamic vital-sign detection system includes a radio frequency (RF) detection device that generates a plurality of detection signals; a correction device that corrects the detection signals; a feature extraction device that processes the corrected detection signals according to at least one feature to obtain a plurality of extraction values and filters out unstable extraction values; and a vital-sign determination device that determines a vital sign according to the extraction values after filtration.

### CROSS-REFERENCE TO RELATED APPLICATIONS

This application claims priority to Taiwan Patent Application No. 108131423, filed on Aug. 30, 2019, the entire contents of which are herein expressly incorporated by reference.

### BACKGROUND OF THE INVENTION

### 1. Field of the Invention

The present invention generally relates to vital-sign detection, and more particularly to a non-contact dynamic vital-sign detection system and method.

### 2. Description of Related Art

Body temperature (BT), blood pressure (BP), heart rate (HR) and respiratory rate (RR) are four primary vital signs. The detection of the vital signs may be used to evaluate health condition and provide a clue to illness of a person.

Conventional health detection devices may be divided into two categories: contact and non-contact. The contact detection device may be worn on the body and may collect vital signs via sensors. The non-contact detection device, such as sensing radar, may obtain vital signs by transmitting radio-frequency (RF) signals and analyzing reflected RF signals.

As the wearable contact detection devices need be worn on the body, their use may be inconvenient or misjudgment may occur due to improper use. The non-contact detection devices may be liable to interference from environmental noise, therefore resulting in misjudgment.

A need has thus arisen to propose a novel scheme to overcome drawbacks of the conventional non-contact health detection devices.

### SUMMARY OF THE INVENTION

In view of the foregoing, it is an object of the embodiment of the present invention to provide a dynamic vital-sign detection method capable of dynamically determining vital signs according to feature extraction of signals, thereby enhancing measurement accuracy.

According to one embodiment, a dynamic vital-sign detection system includes a radio frequency (RF) detection device, a correction device, a feature extraction device and a vital-sign determination device. The RF detection device generates a plurality of detection signals. The correction device corrects the detection signals. The feature extraction device processes the corrected detection signals according to at least one feature to obtain a plurality of extraction values and filters out unstable extraction values. The vital-sign determination device determines a vital sign according to the extraction values after filtration.

### BRIEF DESCRIPTION OF THE DRAWINGS

FIG. 1A shows a block diagram illustrating a dynamic vital-sign detection system according to one embodiment of the present invention;

FIG. 1B shows a detailed block diagram illustrating the correction device of FIG. 1A;

FIG. 1C shows a detailed block diagram illustrating the feature extraction device of FIG. 1A;

FIG. 2 shows a flow diagram illustrating a dynamic vital-sign detection method according to one embodiment of the present invention;

FIG. 3A exemplifies a normal in-phase signal I and a quadrature signal Q;

FIG. 3B exemplifies a normal phase signal P;

FIG. 3C exemplifies an in-phase signal I and a quadrature signal Q with distorted DC level;

FIG. 3D exemplifies a distorted phase signal P;

FIG. 4A shows an exemplary spectrum of a low-pass filter;

FIG. 4B shows exemplary constellation diagrams of an in-phase signal I and a quadrature signal Q;

FIG. 5 exemplifies an in-phase signal I, a quadrature signal Q and a window;

FIG. 6A exemplifies half bandwidth and peak-gain of a signal;

FIG. 6B to FIG. 6E show signals and autocorrelation signals in vital state, motion state, leaving state and no-vital state respectively;

FIG. 7A shows an exemplary signal in different states;

FIG. 7B shows normalized autocorrelation signals corresponding to different states respectively;

FIG. 7C shows autocorrelation signals before normalization in different states respectively;

FIG. 8A shows an exemplary (time-domain) signal in stable state;

FIG. 8B shows an exemplary (time-domain) signal in unstable state;

FIG. 9A exemplifies an in-phase signal I, a quadrature signal Q and fitted curves in stable state; and

FIG. 9B exemplifies an in-phase signal I, a quadrature signal Q and fitted curves in unstable state.

### DETAILED DESCRIPTION OF THE INVENTION

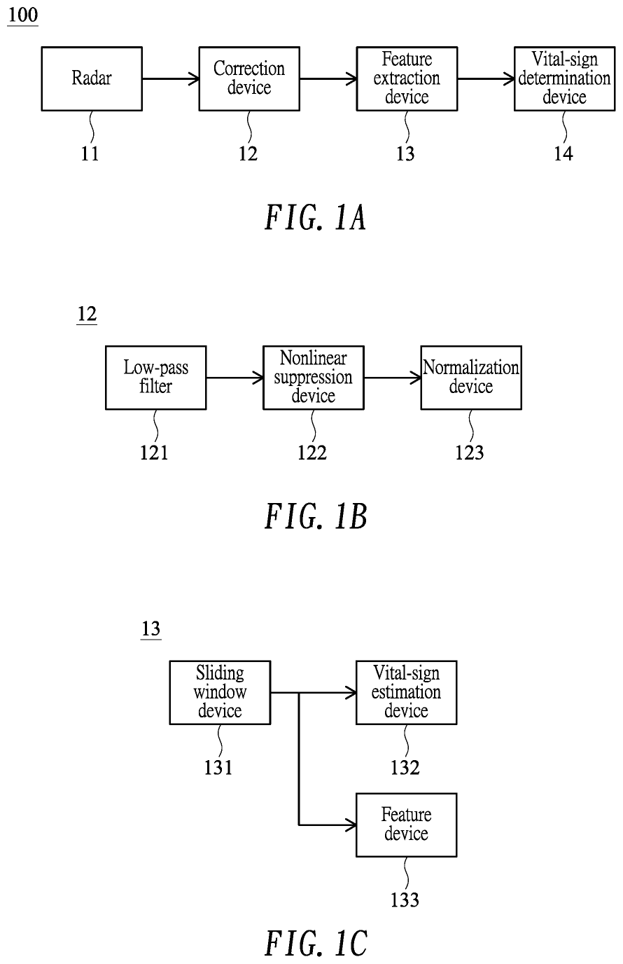

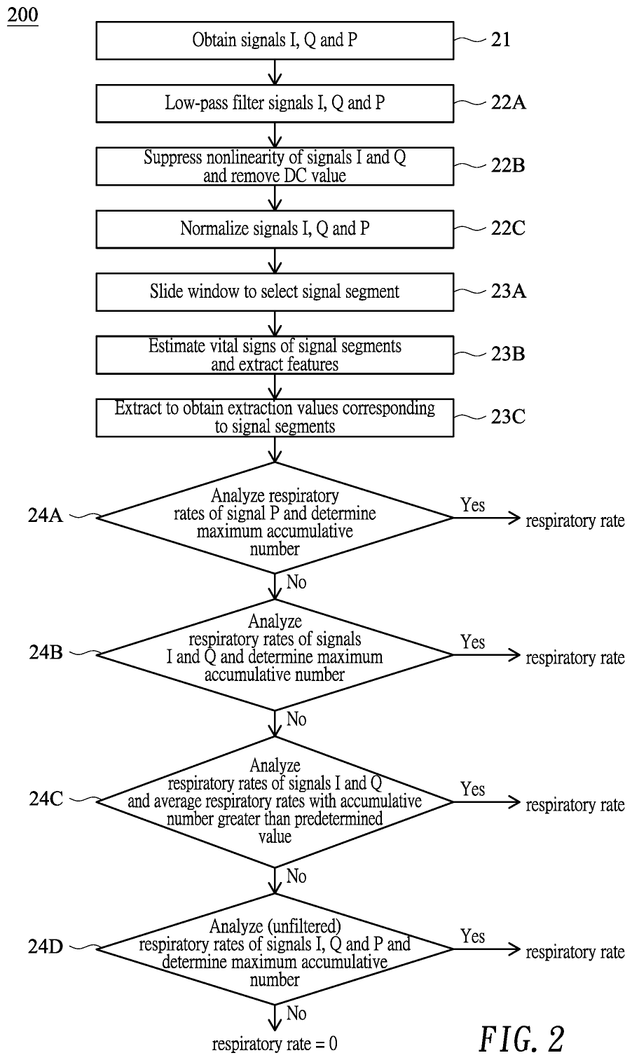

FIG. 1A shows a block diagram illustrating a dynamic vital-sign detection system 100 according to one embodiment of the present invention, and FIG. 2 shows a flow diagram illustrating a dynamic vital-sign detection method 200 according to one embodiment of the present invention. The blocks of FIG. 1A and steps of FIG. 2 may be implemented by hardware, software or their combination. Although the following exemplary embodiment is utilized to detect a respiratory rate, it is appreciated that the embodiment may be utilized to detect other vital signs.

In the embodiment, the dynamic vital-sign detection system (“detection system” hereinafter) 100 may include a radio frequency (RF) detection device, such as a radar 11, configured to generate RF signals to a person under detection and to receive reflected RF signals, which may be converted to obtain detection signals. The detection signal may be decomposed into an in-phase (polarization) signal I, a quadrature (polarization) signal Q and a phase signal P (step 21). Specifically, the phase signal P represents a relative phase of the in-phase signal I and the quadrature signal Q. The radar 11 of the embodiment may be a continuous-wave (CW) radar or an ultra-wideband (UWB) radar (e.g., a frequency modulated continuous waveform (FMCW) radar).

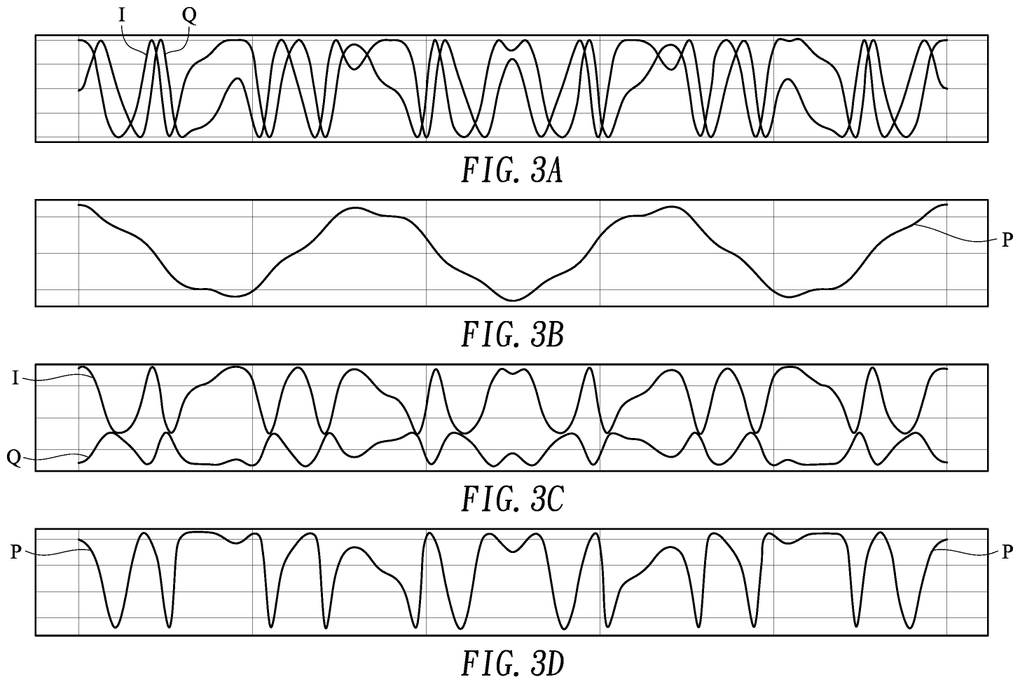

The RF signal is liable to interference from environmental noise to result in nonlinear or time-variant change, which may cause signal distortion of amplitude, phase or direct-current (DC) level. FIG. 3A exemplifies a normal in-phase signal I and a quadrature signal Q, and FIG. 3B exemplifies a normal phase signal P. FIG. 3C exemplifies an in-phase signal I and a quadrature signal Q with distorted DC level. FIG. 3D exemplifies a distorted phase signal P. In this example, the signal period is about 10 seconds, and a respiratory rate of 3 (in 10 seconds) may be estimated according to FIG. 3B. However, a respiratory rate of 12 (in 10 seconds) may be wrongly estimated according to FIG. 3D. Therefore, a scheme of the embodiment is provided to improve this issue.

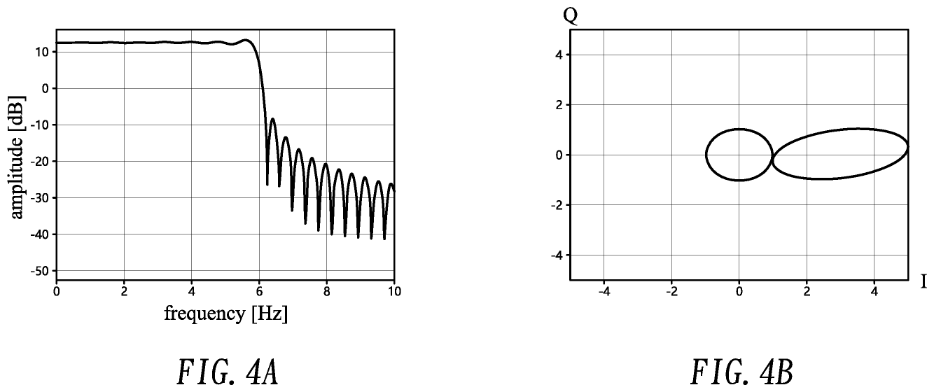

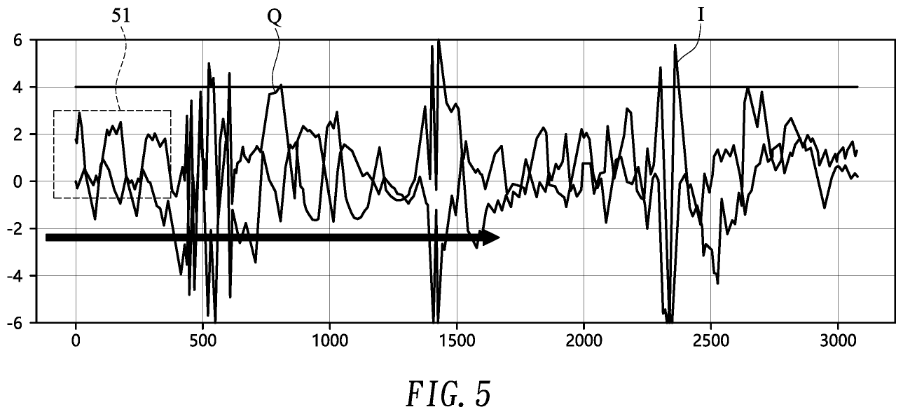

In the embodiment, the detection system 100 may include a correction device 12 configured to correct the in-phase signal I, the quadrature signal Q and the phase signal P in order to eliminate or decrease signal distortion, thereby enhancing signal accuracy. FIG. 1B shows a detailed block diagram illustrating the correction device 12 of FIG. 1A. In the embodiment, the correction device 12 may include a (digital) filter configured to remove unwanted frequency components. The filter of the embodiment may include a low-pass filter 121 configured to pass frequency components of the in-phase signal I, the quadrature signal Q and the phase signal P lower than a cutoff frequency (e.g., 6 Hz) but to attenuate other frequency components (step 22A). FIG. 4A shows an exemplary spectrum of a low-pass filter 121. Generally speaking, the respiratory rate may have an objective range of 0-1 Hz. However, in consideration of succeeding process of the detection system 100 (e.g., feature extraction device 13) that may need extra frequency components, a cutoff frequency higher than a respiratory frequency should be selected. In the embodiment, a cutoff frequency of 6 Hz may be selected. It is appreciated that other cutoff frequency may be selected for different person (e.g., an old person, a child or a middle-aged person having a respiratory rate lower than an infant) under detection. In another embodiment, the detection system 100 may be adapted to detecting a heart rate, and the cutoff frequency should be higher than a heart frequency such that extra frequency components may be used to determine the extent how a signal is affected by environmental noise.

The correction device 12 of the embodiment may include a nonlinear suppression device 122 configure to suppress nonlinear second-harmonic (or above) components of the in-phase signal I and the quadrature signal Q and to remove a direct-current (DC) value thereof (step 22B). FIG. 4B shows exemplary constellation diagrams of an in-phase signal I and a quadrature signal Q. For an ideal in-phase signal I and an ideal quadrature signal Q, the constellation diagram is a circle centered at (0,0). For a distorted in-phase signal I and a distorted quadrature signal Q, the constellation diagram may be an ellipse as shown. In one embodiment, the nonlinear suppression device 122 may adopt a matrix mirroring technique to recover the circular constellation diagram centered at (0,0). At the same time, the nonlinear suppression device 122 may remove the DC value at (0,0).

The correction device 12 of the embodiment may include a normalization device 123 configured to normalize the in-phase signal I, the quadrature signal Q and the phase signal P (step 22C) in order to improve the preceding devices (i.e., the low-pass filter 121 and the nonlinear suppression device 122) or steps (i.e., steps 22A and 22B) that improperly scale the signals.

In the embodiment, the detection system 100 may include a feature extraction device 13 configured to process the corrected in-phase signal I, the quadrature signal Q and the phase signal P according to at least one feature to obtain extraction values and to filter out (or screen) unstable extraction values. FIG. 1C shows a detailed block diagram illustrating the feature extraction device 13 of FIG. 1A. The feature extraction device 13 of the embodiment may include a sliding window device 131 having a predetermined window size (e.g., 10 seconds) configured to select a signal segment in time order (step 23A). FIG. 5 exemplifies an in-phase signal I and a quadrature signal Q, on which a window 51 with a size of 10 seconds slides rightwards (as indicated by the arrow) every 2.5 seconds. Therefore, 21 signal segments may be selected in one minute.

The feature extraction device 13 of the embodiment may include a vital-sign estimation device 132 configured to estimate (initial) vital signs corresponding to the signal segments respectively and extract features (step 23B). In the embodiment, the vital-sign estimation device 132 may adopt a zero-crossing rate method to estimate a respiratory rate by counting crossings between a signal and a zero DC level. As two crossings indicate one respiration, the respiratory rate may be obtained by dividing the amount of crossings by two.

The feature extraction device 13 of the embodiment may include a feature device 133 configured to obtain extraction values corresponding to the signal segments respectively according to at least one feature (step 23C), and to filter out unstable extraction values (and corresponding vital signs) according to a predetermined threshold. The feature extraction device 13 of the embodiment may perform feature extraction according to one or more of the following features: half bandwidth, peak-gain, kurtosis, root mean square (RMS), standard deviation (STD) and peak-to-peak difference (Vpp).

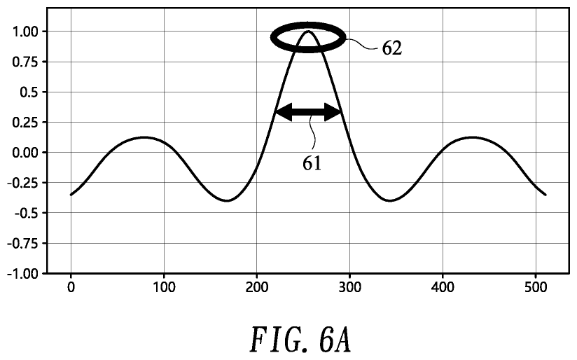

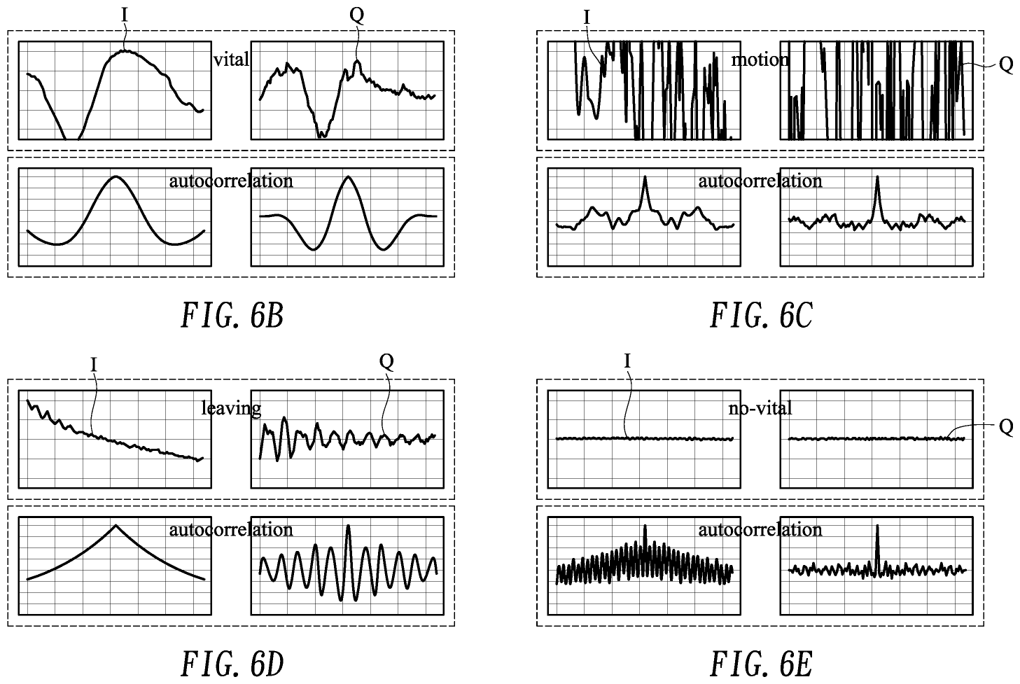

FIG. 6A exemplifies half bandwidth 61 and peak-gain 62 of a signal. FIG. 6B to FIG. 6E show signals (e.g., in-phase signal I and quadrature signal Q) and autocorrelation signals in vital state (e.g., rest or sleep), motion state, leaving state and no-vital state (e.g., left) respectively. According to the signals as shown, stable signals (e.g., FIG. 6B) may have larger half bandwidth; unstable signals in motion state (e.g., FIG. 6C) may have larger peak-gain; and unstable signals in no-vital state (e.g., FIG. 6E) may have less peak-gain. Accordingly, the feature device 133 may filter out unstable extraction values (and corresponding vital signs) according to a predetermined threshold.

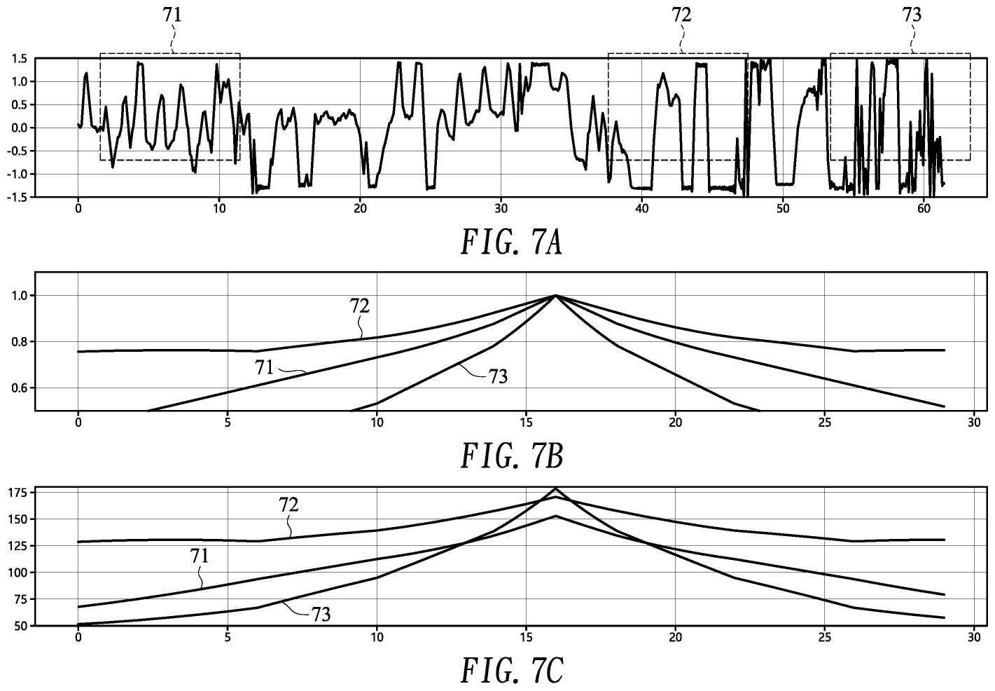

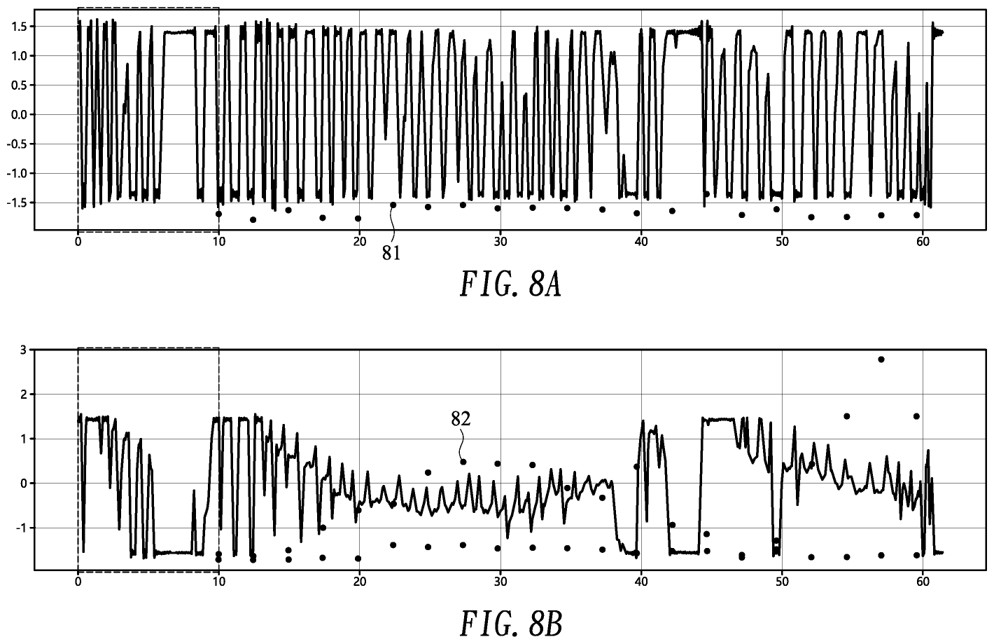

FIG. 7A shows an exemplary signal in different states 71, 72 and 73. FIG. 7B shows normalized autocorrelation signals corresponding to different states 71, 72 and 73 respectively. Specifically, the half bandwidth (0.165) in (stable) state 71 is larger than the half bandwidth (0.106) in (unstable) state 72, but is less than the half bandwidth (0.282) in another (unstable) state 73. FIG. 7C shows autocorrelation signals before normalization in different states 71, 72 and 73 respectively. Specifically, the peak-gain (153) in (stable) state 71 is less than the peak-gain (170) in (unstable) state 72, and is less than the peak-gain (178) in another (unstable) state 73. Accordingly, in the embodiment, the feature device 133 may separate the states 71, 72 and 73 into stable states and unstable states, and then filter out unstable extraction values (and corresponding vital signs) according to a predetermined threshold.

FIG. 8A shows an exemplary (time-domain) signal in stable state having lower kurtosis 81, and FIG. 8B shows an exemplary (time-domain) signal in unstable state apparently having higher kurtosis 82. Accordingly, the feature device 133 may determine stability according to kurtosis. The kurtosis K may be defined as follows:

$$K = \frac{{{\sum }_{i = 1}}^{m} {( {x}_{i} - \overline{x} )}^{4}}{n {s}^{4}} - 3$$

where xᵢ represents an i-th measurement value, s represents a standard deviation, n represents a sample size, and x̅ represents an arithmetic mean.

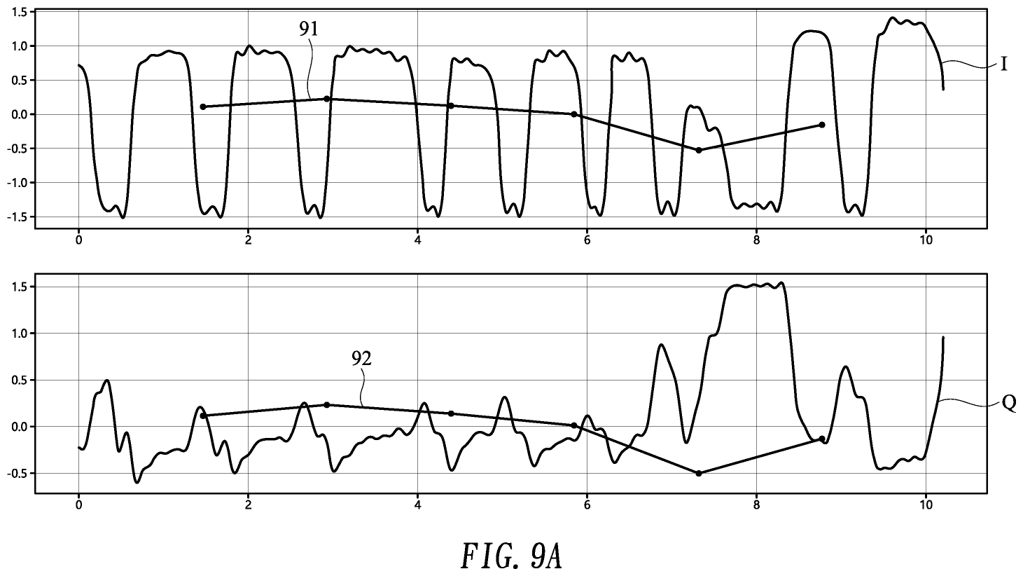

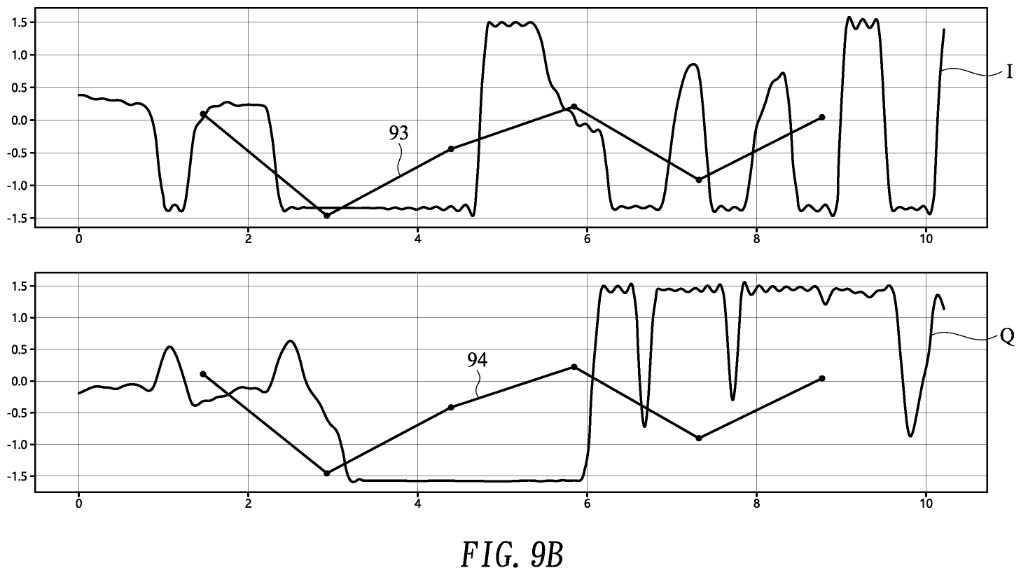

FIG. 9A exemplifies an in-phase signal I and a quadrature signal Q in stable state. A polynomial fitting method may be adopted to construct a fit to a DC level of the in-phase signal I and the quadrature signal Q, thereby obtaining fitted curves 91 and 92. FIG. 9B exemplifies an in-phase signal I and a quadrature signal Q in unstable state. A polynomial fitting method may be adopted to construct a fit to a DC level of the in-phase signal I and the quadrature signal Q, thereby obtaining fitted curves 93 and 94. According to the fitted curves as shown, fitted curves 91 and 92 in stable stage approximate straight lines representing zero DC level; and the fitted curves 93 and 94 in unstable stage disturb and are distant from zero DC level. Accordingly, unstable extraction values (and corresponding vital signs) may be filtered out according to root mean square (RMS), standard deviation (STD) or peak-to-peak difference (Vpp) obtained from the fitted curves. The root mean square M, standard deviation SD or peak-to-peak difference Vₚₚ may be defined as follows:

$$M = \sqrt{\frac{{\sum }_{i = 1}^{n} {x}_{i}^{2}}{n}}$$

$$SD = \sqrt{\frac{1}{n} {\sum }_{i = 1}^{n} {( {x}_{i} - \overline{x} )}^{2}}$$

$${V}_{pp} = max ( {x}_{i} ) - min ( {x}_{i} )$$

where xᵢ represents an i-th measurement value, n represents a sample size, x̅ represents an arithmetic mean, max( ) represents a maximum function, and min( ) represents a minimum function.

The detection system 100 of the embodiment may include a vital-sign determination device 14 configured to determine a (final) vital sign (e.g., respiratory rate) according to the extraction values (from the feature device 133) corresponding to the signal segments and the corresponding (initial) vital signs (from the vital-sign estimation device 132). The correction device 12, the feature extraction device 13 and the vital-sign determination device 14 may be distinct signal processing devices respectively. Alternatively, two or all of the correction device 12, the feature extraction device 13 and the vital-sign determination device 14 may be integrated into a single signal processing device.

In step 24A, (e.g., 21 pieces of) the (initial) respiratory rates (corresponding to the signal segments) of the phase signal P are statistically analyzed, among which the respiratory rate corresponding to a maximum accumulative number of the respiratory rate is outputted as the (final) respiratory rate, where the maximum accumulative number should be greater than a first predetermined value (e.g., 2). The rationale of analyzing the phase signal P in the first step (i.e., step 24A) of the vital-sign determination device 14 is that the phase signal P commonly has a better effect on nonlinear suppression.

If step 24A cannot determine the respiratory rate, the flow goes to step 24B to statistically analyze (e.g., 42 pieces of) the (initial) respiratory rates (corresponding to the signal segments) of the in-phase signal I and the quadrature signal Q, thereby determining the respiratory rate with a maximum accumulative number and outputting the determined respiratory rate as the (final) respiratory rate, where the maximum accumulative number should be greater than a second predetermined value (e.g., 3).

If step 24B cannot determine the respiratory rate, the flow goes to step 24C to statistically analyze (e.g., 42 pieces of) the (initial) respiratory rates (corresponding to the signal segments) of the in-phase signal I and the quadrature signal Q, and to average all respiratory rates with an accumulative number greater than a third predetermined value (e.g., 3), thereby obtaining an average value outputted as the (final) respiratory rate.

It is noted that the respiratory rates in steps 24A-24C are respiratory rates after filtering out unstable extraction values. If step 24C cannot determine the respiratory rate, the flow goes to step 24D, in which respiratory rates before filtering out unstable extraction values are used. In step 24D, (e.g., 63 pieces of) the (initial) respiratory rates (corresponding to the signal segments) of the in-phase signal I, the quadrature signal Q and the phase signal P are statistically analyzed, among which the respiratory rate greater than a predetermined apnea threshold (e.g., 9) and corresponding to a maximum accumulative number of the respiratory rate is outputted as the (final) respiratory rate, where the maximum accumulative number should be greater than a fourth predetermined value (e.g., 24); or alternatively the respiratory rate not greater than the predetermined apnea threshold and corresponding to a maximum accumulative number of the respiratory rate is outputted as the (final) respiratory rate, where the maximum accumulative number should be greater than a fifth predetermined value (e.g., 12). The fifth predetermined value is ordinarily less than the predetermined fourth value. If step 24D cannot determine the respiratory rate, the respiratory rate of zero is outputted.

Although specific embodiments have been illustrated and described, it will be appreciated by those skilled in the art that various modifications may be made without departing from the scope of the present invention, which is intended to be limited solely by the appended claims.

What is claimed is:

1. A dynamic vital-sign detection system, comprising:
a radio frequency (RF) detection device that generates a plurality of detection signals;
a correction device that corrects the detection signals;
a feature extraction device that processes the corrected detection signals according to at least one feature to obtain a plurality of extraction values and filters out unstable extraction values; and
a vital-sign determination device that determines a vital sign according to the extraction values after filtration.

2. The system of claim 1, wherein the correction device comprises:
a filter that removes unwanted frequency components of the detection signals;
a nonlinear suppression device that suppresses nonlinear components of the detection signals; and
a normalization device that normalizes the detection signals.

3. The system of claim 1, wherein the feature extraction device comprises:
a sliding window device that selects signal segments of the detection signals to be processed in time order according to a predetermined window size;
a vital-sign estimation device that estimates initial vital signs corresponding to the signal segments respectively; and
a feature device that obtains extraction values corresponding to the signal segments respectively according to at least one feature, and filters out unstable extraction values according to a predetermined threshold.

4. The system of claim 3, wherein the vital-sign estimation device adopts a zero-crossing rate method to estimate the initial vital signs.

5. The system of claim 3, wherein the detection signal is decomposed into an in-phase signal, a quadrature signal and a phase signal.

6. The system of claim 5, wherein the vital-sign determination device performs the following steps:
(a) statistically analyzing the initial vital signs corresponding to the signal segments of the phase signal, among which an initial vital sign corresponding to a maximum accumulative number, which is greater than a first predetermined value, is outputted as the vital sign;
(b) if the step (a) cannot determine the vital sign, statistically analyzing the initial vital signs corresponding to the signal segments of the in-phase signal and the quadrature signal, among which an initial vital sign corresponding to a maximum accumulative number, which is greater than a second predetermined value, is outputted as the vital sign;
(c) if the step (b) cannot determine the vital sign, statistically analyzing the initial vital signs corresponding to the signal segments of the in-phase signal and the quadrature signal, and averaging all vital signs with an accumulative number greater than a third predetermined value, thereby obtaining an average value outputted as the vital sign; and
(d) if the step (c) cannot determine the vital sign, statistically analyzing the initial vital signs corresponding to the signal segments of the in-phase signal, the quadrature signal and the phase signal, among which a vital sign greater than a predetermined apnea threshold and corresponding to a maximum accumulative number, which is greater than a predetermined fourth predetermined value, is outputted as the vital sign; or alternatively a vital sign not greater than the predetermined apnea threshold and corresponding to a maximum accumulative number, which is greater than a predetermined fifth predetermined value, is outputted as the vital sign;
wherein the initial vital signs in the steps (a) to (c) are initial vital signs after the unstable extraction values are filtered out by the feature device, but the initial vital signs in the step (d) are initial vital signs before the unstable extraction values are filtered out by the feature device.

7. The system of claim 1, wherein the at least one feature comprises one or more of the following features: half bandwidth, peak-gain, kurtosis, root mean square (RMS), standard deviation (STD) and peak-to-peak difference (Vpp).

8. The system of claim 7, wherein the feature extraction device adopts a polynomial fitting method to construct a fit to a direct-current (DC) level of the detection signals, thereby obtaining fitted curves.

9. The system of claim 1, wherein the vital signal comprises respiratory rate.

10. A dynamic vital-sign detection method, comprising:
(I) generating a plurality of detection signals;
(II) correcting the detection signals;
(III) processing the corrected detection signals according to at least one feature to obtain a plurality of extraction values and filtering out unstable extraction values; and
(IV) determining a vital sign according to the extraction values after filtration.

11. The detection method of claim 10, wherein the step (II) comprises:
(IIa) removing unwanted frequency components of the detection signals;
(IIb) suppressing nonlinear components of the detection signals; and
(IIc) normalizing the detection signals.

12. The detection method of claim 11, wherein the step (IIa) comprises:
passing frequency components of the detection signals lower than a cutoff frequency but attenuating other frequency components, the cutoff frequency being higher than a respiratory frequency.

13. The detection method of claim 10, wherein the step (III) comprises:
(IIIc) selecting signal segments of the detection signals to be processed in time order according to a predetermined window size;
(IIIb) estimating initial vital signs corresponding to the signal segments respectively; and
(IIIc) obtaining extraction values corresponding to the signal segments respectively according to at least one feature, and filtering out unstable extraction values according to a predetermined threshold.

14. The detection method of claim 13, wherein the step (IIIb) comprises:
adopting a zero-crossing rate method to estimate the initial vital signs.

15. The detection method of claim 13, wherein the detection signal is decomposed into an in-phase signal, a quadrature signal and a phase signal.

16. The detection method of claim 15, wherein the step (IV) comprises:
(IVa) statistically analyzing the initial vital signs corresponding to the signal segments of the phase signal, among which an initial vital sign corresponding to a maximum accumulative number, which is greater than a first predetermined value, is outputted as the vital sign;
(IVb) if the step (IVa) cannot determine the vital sign, statistically analyzing the initial vital signs corresponding to the signal segments of the in-phase signal and the quadrature signal, among which an initial vital sign corresponding to a maximum accumulative number, which is greater than a second predetermined value, is outputted as the vital sign;
(IVc) if the step (IVb) cannot determine the vital sign, statistically analyzing the initial vital signs corresponding to the signal segments of the in-phase signal and the quadrature signal, and averaging all vital signs with an accumulative number greater than a third predetermined value, thereby obtaining an average value outputted as the vital sign; and
(IVd) if the step (IVc) cannot determine the vital sign, statistically analyzing the initial vital signs corresponding to the signal segments of the in-phase signal, the quadrature signal and the phase signal, among which a vital sign greater than a predetermined apnea threshold and corresponding to a maximum accumulative number, which is greater than a predetermined fourth predetermined value, is outputted as the vital sign; or alternatively a vital sign not greater than the predetermined apnea threshold and corresponding to a maximum accumulative number, which is greater than a predetermined fifth predetermined value, is outputted as the vital sign;
wherein the initial vital signs in the steps (IVa) to (IVc) are initial vital signs after the unstable extraction values are filtered out in the step (III), but the initial vital signs in the step (IVd) are initial vital signs before the unstable extraction values are filtered out in the step (III).

17. The detection method of claim 10, wherein the at least one feature comprises one or more of the following features: half bandwidth, peak-gain, kurtosis, root mean square (RMS), standard deviation (STD) and peak-to-peak difference (Vpp).

18. The detection method of claim 17, wherein the step (III) adopts a polynomial fitting method to construct a fit to a direct-current (DC) level of the detection signals, thereby obtaining fitted curves.

19. The detection method of claim 10, wherein the vital signal comprises respiratory rate.
